(19) United States
(12) Patent Application Publication
(10) Pub. No.: US 2013/0189364 A1
(43) Pub. Date: Jul. 25, 2013

(54) COMPOSITIONS AND METHODS OF POTENTIATING ADJUVANT PHARMACEUTICALS TARGETING LATENT VIRAL INFECTIONS

(71) Applicant: Robert Sabin, Mill Neck, NY (US)

(72) Inventor: Robert Sabin, Mill Neck, NY (US)

(21) Appl. No.: 13/801,470

(22) Filed: Mar. 13, 2013

Related U.S. Application Data
(63) Continuation-in-part of application No. 12/152,752, filed on May 16, 2008.
Application No. 12/152,752 is a division of application No. 10/888,576, filed on Jul. 9, 2004, now Pat. No. 7,449,196.
Application No. 10/888,576 is a continuation-in-part of application No. 11/891,613, filed on Aug. 10, 2007, now abandoned.
Application No. 11/891,613 is a continuation of application No. 10/888,576, filed on Jul. 9, 2004, now Pat. No. 7,449,196.
Application No. 10/888,576 is a continuation-in-part of application No. 11/192,752, filed on Jul. 29, 2005.
Application No. 11/192,752 is a continuation-in-part of application No. 10/888,576, filed on Jul. 9, 2004, now Pat. No. 7,449,196.
(60) Provisional application No. 60/666,135, filed on Mar. 29, 2005.
(60) Provisional application No. 60/598,179, filed on Aug. 2, 2004.

(30) Foreign Application Priority Data
Jul. 8, 2005 (US) PCT/US05/24272

(51) Int. Cl. A61K 9/50

(52) U.S. Cl. CPC A61K 9/5089
USPC 424/490; 424/278.1; 424/281.1

(57) ABSTRACT

A composition and method for potentiating, sensitizing, and/or amplifying at least one adjuvant pharmaceutical targeting at least one latent viral infection in a patient is provided. In one embodiment, the composition is administered to potentiate, sensitize and/or amplify an adjuvant pharmaceutical targeting at least one latent viral infection such as those which are currently being investigated for use with anti-HIV drugs/antiretrovirals HAART.

### RELATED APPLICATIONS

This application is a continuation-in-part of application Ser. No. 12/152,752 filed May 16, 2008; continuation-in-part of application Ser. No. 11/891,613 filed Aug. 10, 2007; continuation-in-part of application Ser. No. 11/192,752 filed Jul. 29, 2005 and Published as U.S. Patent Application Publication No. 2006/0147512 A1 on Jul. 6, 2006; and continuation-in-part of application Ser. No. 10/888,576 filed Jul. 9, 2004, now U.S. Pat. No. 7,449,196 issued Nov. 11, 2008; and claims priority under 35 U.S.C. 120 therefrom. This application is also based in part upon provisional application No. 60/598,179 filed on Aug. 2, 2004 and upon provisional application No. 60/666,135, filed on Mar. 29, 2005, and claims benefit under 35 U.S.C. 119(e) therefrom. This application is also based in part upon PCT/US05/24272 and claims benefit under 35 U.S.C. 119(b) therefrom. The content of each application is expressly incorporated herein by reference.

### TECHNICAL FIELD

The present disclosure relates generally to a composition and method for potentiating, sensitizing, and/or amplifying at least one adjuvant pharmaceutical targeting at least one latent viral infection in a patient.

### BACKGROUND OF THE INVENTION

All patents, scientific articles and other documents mentioned herein are expressly incorporated by reference as if reproduced in full.

The HIV-1 pandemic has claimed over 20 million lives, with 38.6 million people worldwide currently infected, and will continue to be a significant global health problem as there is no vaccine available. Currently, the only effective treatment available to HIV-1 infection is HAART (highly active antiretroviral therapy), which has led to a profound reduction in HIV-1 related morbidity and mortality. However, HAART fails to eliminate the virus in vivo, mainly due to the persistent existence of long lived latently infected cells harboring replication-competent proviruses. Efforts to purge these infected cells have focused on reactivation of the proviruses. It is presumed that these infected cells will be killed after reactivation of virus gene expression by viral cytopathic effects (CPEe), host immune responses or both. Several proviral stimulants, also referred to herein as adjuvant pharmaceuticals, including IL-7, valpoic acid (VPA), suberoylanilide hydroxamic acid (SAHA) and prostatin have been explored to force activation of proviruses in latently infected resting CD4+ T cells that constitute the major reservoir of HIV-1 in vivo. However, treatment with IL-7 or VPA in patients on HAART has failed to reduce HIV-1 latency, suggesting that these agents alone are not sufficient to induce killing of latently infected cells. Therefore, there is a need to develop an “activation-killing” approach by priming pre-treatment of latently infected cells with adjuvant agents that will increase, amplify, sensitize and potentiate the cytopathic potential cell killing effect of these adjuvant proviral stimulants. HIV latency and HIV pathogenesis, dynamics and genetics of viral populations and infected cells and viral sanctuary sites are disclosed by John Coffin in “HIV Pathogenesis: Dynamics and Genetics of Viral Populations and Infected Cells, Cold Spring Harb Perspect Med 2013.3:, www.perspectivesinmedicine.org/content/3/1/a012526.full. HIV latency is disclosed by Robert F. Siliciano in “HIV Latency”, Cold Spring Harb Perspect Med 2011.1, www.perspectivesinmedicine.org/content/1/1/a007096.full. This proviral latency is thought to be the chief stumbling block for eradication of the virus or a functional cure taken together with the viral sanctuary sites where there is poor drug trafficking or penetration or crossing the blood/brain barrier. Because it is thought that as few as one in one million CD4+ memory cells are latently infected, targeting this specific cell population is extremely difficult. (See Liz Highleyman, “HIV Eradication: Time to Talk About a Cure”, BET, Winter/Spring 2011, pages 13-27) Researchers say that it has been proven all but impossible to wipe out these pockets of infection. (See Health and Medicine, “To Kill Latent HIV, Lure into an Ambush”, Futurity.org, Mar. 16, 2012) From the San Francisco Business Times: http://www.bizjournals.com/sanfrancisco/blog/biotech/2012/11/tony-fauci-hiv-truvada-gilead.html, Dr. Tony Fauci, the nation's HIV research general, discloses, “This is what viruses usually do: They come in, they bind, they enter, they go in, they integrate and then they come out and bud out to more. So, when a cell is latent, it goes in but it never buds out; it just stays latent. So, the question is, if you're going to attack this cell that the only thing the body sees is a cell that otherwise looks normal. Can you develop a probe that's safe, that can go in and recognize that this pro-virus is the coding sequence for HIV and whenever it sees that it essentially kills that cell? Now that means that you've got to put something into somebody that's got to be able to permeate every cell. That is not easy. I don't even know if this is the right pathway, but you're talking about some seriously difficult things to do.” Therefore, one may question whether it is the ultimate goal with these reservoirs of latently infected cells then to dry them up and eradicate them or keep them at bay? Fauci says: “You can either purge it—and you can purge it by activating it and killing the cell after it spits it out, and then it dies. We tried to do that: The cells spit it out but it never died. So we said, ‘Hmmm, it doesn't work’. “Now there are other experiments that are trying to suppress that and get rid of it. These are ongoing experiments.” (see San Francisco Business Times: One-on-One Nation's HIV research general Tony Fauci: Cure in ‘discovery phase’, 12 Nov. 2012). Fauci says: “The other one is, saying we're not going to get rid of it; we're going to do what's called a “functional cure.” There are two ways to do that: either enhance immunity, in other words if I get someone with very, very low level of reservoir and maybe give a vaccine or a passive infusion of antibodies that can keep it down so that you didn't eradicate it but the bodies immune system (keeps it at bay). That's tough, because under natural conditions, the body doesn't do that. It's kind of tough to get the body to do something it wouldn't do under natural conditions.” (see San Francisco Business Times: One-on-One Nation's HIV research general Tony Fauci: Cure in ‘discovery phase’, 12 Nov. 2012). This approach, which doesn't work, is known as “kick and kill” (See Gus Cairns, “Eliminating HIV: latest progress”, HIV & AIDS Information: Issue 211: Spring 2012, pgs. 1-3) or “shock and kill”. (See Steven G. Deeks, “Shock and Kill”, New & Views Research, Nature, Vol 487, Jul. 26, 2012, pgs. 439-440)

The only problem with this approach, as Dr. Fauci said, is that we are capable of kicking, but not killing the provirally infected cell. Moreover, as Dr. Fauci discloses, “But you're talking about some seriously difficult things to do.” It is generally believed that unless you inactivate, disable, kill these latently infected cells throughout the body and sanctuary sites that there never will be a cure for AIDS as this low level, viral replication will re-infect the body upon cessation of HAART drugs. Possibly immunotherapy may provide a functional cure. So far it hasn't.

Normally, an infected cell dies after virus is produced within it. But in this case, the damage caused by provoking HIV growth with SAHA/vorinostat/HDAC inhibitor was not sufficient to kill the cells and thereby remove their reservoir potential. This is a profound disappointment. (Laurence, Jeffrey, “Destroying HIV Reservoirs”, AMFAR, 11 Apr. 2012).

In recent years, antiviral drugs have reached the limit of their effectiveness. The cost of providing universal access estimated at $24 billion in 2015 has become unsustainable. Evidence underscores the detrimental effects of persistent HIV infection even while plasma viral load is low and CD4 cells count high. For every person starting treatment another two are newly infected and only a fraction of the 34 million HIV positive patients are under treatment. (see “HIV Eradication: Time to Talk About a Cure”, The BodyPro.com, Spring 2012) (See Szabo, Liz, “Science sets its sights again on an AIDS cure”, USATODAY.com, 10 Jul. 2012)

Current Anti-HIV drugs, FDA approved, are disclosed in the following publication: United States Department of Veterans Affairs, Treatment Decisions for HIV, www.hiv.va.gov/patient/treat/decisions-single-page.asp.

Margolis, David M., “Searching for a Cure”, HIV Specialist cover story, December 2012, discloses Histone Deacetylase (HDACis) inhibitors as anti-latency drugs. Disulfiram and PD-1 inhibitors are other agents being investigated to activate latent HIV infection along with other pharmaceuticals and methods and vaccination to activate latent HIV in the “kick and kill” approach disclosed by Margolis.

The challenges are many. For an eradication therapy to be effective and feasible for worldwide use, it must:*be safe and have manageable side effects;*not extensively activate the immune system, as activated T cells are more susceptible to HIV infection and are more difficult to protect with antiretroviral therapy;*have a finite duration that will allow the patient to live a healthy life without ongoing treatment;*be able to access all reservoirs of persistent infection throughout the body;*be economically and logistically accessible to the developing world.

There has been one person in the history of the world, who has been cured of HIV/AIDS—this is “the Berlin patient”—one patient out of 34 million currently affected and 20 million fatalities. His treatment is unreachably expensive, difficult, grueling, toxic and unsuitable for expansion. Clearly there is room for improvement for a scalable, druggable and cheapable (cost effective) and brainable (capable of being administered without straining the mental capacity of the medical team) eradication or functional cure therapy. Iron and copper can damage DNA (see Sagripanti, DNA Damage Mediated by Metal Ions with Special Reference to Copper and Iron) and inactivate HIV (See Sagripanti, “Cupric and Ferric Ion Inactivate HIV”, AIDS Research and Human Retroviruses, Vol. 12, No. 4, 1996, pgs. 333-336).

Iron dextran has been administered since 1955 intramuscularly and IV since 1971. There have been hundreds of millions of administrations in the United States and throughout the world. All intravenous (IV) iron agents are colloids that consist of spheroidal iron-carbohydrate nanoparticles. At the core of each particle is an iron-oxyhydroxide gel. The core is surrounded by a shell of carbohydrate that stabilizes the iron-oxyhydroxide, slows the release of bioactive iron, and maintains the resulting particles in colloidal suspension. (see Danielson, “Structure, Chemistry, and Pharmacokinetics of Intravenous Iron Agent”, J Am Soc Nephrol 15:93-98, 2004)

Moreover, after IV administration, iron dextran mixes with plasma and then enters the RES system directly from the intravascular fluid department. Iron dextran, in the plasma, crosses the BB barrier, the bone marrow, the lymphatic system and anywhere plasma traffics, iron dextran traffics, which is everywhere. (see Danielson) Iron dextran also is taken up by red blood cells (in vitro activity against malaria with copper drug) which means dextran is taken up by RBC.

In contrast to other parenteral iron preparations, because of the core/shell configuration of iron dextran, it has been given intravenously in doses as high as 2-3 gm without apparent toxicity from the release of excessive free iron into the circulation. The reticuloendothelial system plays a key role in the utilization of the remaining portion of the iron dextran complex. After an intravenous administration of large doses, 2000-3000 mg of the iron dextran complex, complete removal of the material from plasma takes as long as 2-3 weeks. With infusions of up to 500 mg of the material at one time, clearance into the reticuloendothelial system is exponential. When doses in excess of 500 mg are administered, the initial clearance rate does not exceed 10-20 mg/hr., the maximum removal rate of the reticuloendothelial system. Once cleared, the material is readily visible as iron stores on Prussian blue stain of marrow stroma. (see Henderson & Hillman, “Characteristics of Iron Dextran Utilization in Man”, BLOOD, Vol. 34, No 3 (September) 1969, pgs. 357-370) (see Marchasin & Wallerstein, “The Treatment of Iron-Deficiency Anemia with Intravenous Iron Dextran”, BLOOD, Vol. 23, No. 3 (March), 1961) Because iron dextran is a core/shell formulation, with the dextran chains encapsulating the beta ferric oxyhydroxide core, the cellular toxicity of iron is severely curtailed so that doses many times that of iron salts may be administered safely. A fraction of a common three-gram dose of iron dextran IV using iron salts is fatal. There are thousands of papers published about iron dextran. It has been used extensively with kidney dialysis and cancer and more. Published experience with more than 1000 patients in clinical trials in oncology alone involving the use of IV Fe suggests minimal toxicity and substantial benefits are experienced when high molecular weight iron dextran is avoided. (see Auerbach, “Intravenous Iron Optimizes the Response to Recombinant Human Erythopoietin in Cancer Patients with Chemotherapy-Related Anemia: A Multicenter, Open-Label, Randomized Trial”, Journal of Clinical Oncology, Vol. 22, No. 7, Apr. 1, 2004, pgs. 1301-7) Moreover, 41 cancer patients were administered total dose infusion of iron dextran from 1000 mg/one gram to 3000 mg/three grams with little toxicity. (See Auerbach, JCO)

Iron dextran is also used for livestock throughout the world and is FDA approved for this purpose. Iron dextran is generic and with the human FDA approved version INFED® costing about 100 times more than the veterinary version. The previous PK studies from Henderson & Hillman and Marchasin used IMFERON®, which is no longer available. The current FDA approved iron dextran INFED® is far more robust with a longer plasma half-life than IMFERON®. (see Table 3, U.S. Pat. No. 5,624,668 of Lawrence et.al.) U.S. Pat. No. 5,202,353 of Schroth discloses that iron added to cupric hydroxide increases the antibacterial effect of cupric hydroxide in vitro and reverses copper resistance of bacteria restoring high activity. (See FIGS. 1A and 1B of U.S. Pat. No. 5,202,353) Moreover, Schroth further discloses the addition of added iron increases activity. The more iron added, the more activity. U.S. Patent Application Publication 20060147512 to Sabin discloses antitumor activity in mouse xenographs with lung and breast tumors in mice with intratumoral injection of copper dextran without toxicity and generation of ROS. Sabin further discloses the results of the well known NCI60 human tumor cell line anti cancer drug screen. Iron dextran in all cases adds to the cytotoxicity of the copper dextran so that less copper is required to achieve an IC50 in all cases. Moreover, the same result is obtained in the IC90 with less copper being required to achieve an IC90 in all cell lines. Moreover, the addition of iron further increased the IC100 in many cell lines. The addition of iron also reversed resistance to copper in some cell lines to IC50's, IC90's, and IC100's which did not achieve any of the aforementioned IC's without the addition of added iron (see Ang Sun, Robert Sabin et al, “Cupric hydroxide-dextran induces cancer cell death both in vitro and in vivo through ROS generation and activation of the intrinsic apoptosis pathway)

As provided in U.S. Patent Application Publication 20060147512 of Sabin, applicant has administered iron dextran to Cyno Mulgus monkeys at 400 and 500 mg/kg of body weight without toxicity. Moreover, the publication discloses robust, anti HIV activity with copper dextran against multiple isolates of HIV infection in Peripheral Blood Mononuclear Cells (PBMCs) in vitro.

### SUMMARY OF THE INVENTION

This disclosure relates to a method for potentiating, sensitizing, and/or amplifying at least one adjuvant pharmaceutical targeting at least one latent viral infection in a patient by forming a composition including a colloidal solution having a core of at least a biologically acceptable fixed copper compound or a biologically acceptable insoluble iron compound or mixtures thereof wherein said core is encapsulated, encoated, adsorbed, complexed or bound in at least one of a sheath, a shell, a polymeric shell, a cover, a casing, an encoating, a jacket or combination thereof, and a pharmaceutically acceptable carrier. By “fixed copper compound”, the term is defined to mean that the copper compound is insoluble or highly insoluble in water. See Richardson, “Manufacture of Copper Compounds”, Handbook of Copper Compounds and Applications, p. 55-89, and Rosenberger, “Options, Benefits and Liabilities For Copper Sprays in Tree Fruiting,” Scaffold's Fruit Journal, Vol. 21, No. 1, Mar. 12, 2012, p. 6. The sheath, shell, polymeric shell, cover, casing, encoating, jacket or combination thereof prevents immediate chemical interaction of the core with the surrounding environment. The composition is administered to the patient to potentiate, sensitize and/or amplify the at least one adjuvant pharmaceutical targeting the at least one latent viral infection in the patient. In one embodiment, the composition is administered to potentiate, sensitize and/or amplify an adjuvant pharmaceutical targeting at least one latent viral infection such as those which are currently being investigated for use with anti-HIV drugs/antiretrovirals HAART. (See United States Department of Veterans Affairs, Treatment Decisions for HIV). Adjuvants pharmaceuticals are agents that aid or increase the action of the principal drug or that affect the absorption, mechanism of action, metabolism, or excretion of the primary drug in such a way as to enhance its effects.

This disclosure also relates to a chemical formulation for potentiating, sensitizing, and/or amplifying adjuvants pharmaceuticals used in conjunction with antiretroviral therapy comprising a colloidal solution having a core of at least a biologically acceptable fixed copper compound or a biologically acceptable insoluble iron compound or mixtures thereof wherein said core is encapsulated, encoated, adsorbed, complexed or bound in at least one of a sheath, a shell, a polymeric shell, a cover, a casing, an encoating, a jacket or combination thereof, and a pharmaceutically acceptable carrier. The sheath, shell, polymeric shell, cover, casing, encoating, jacket or combination thereof prevents immediate chemical interaction of the core with the surrounding environment. This is known as a core/shell configuration in the art and is also disclosed in U.S. Pat. No. 7,449,196 to Sabin, the contents of which are expressly incorporated herein by reference. In one embodiment, the chemical composition is administered to patients taking at least one proviral stimulant with or without conventional ART/HAART treatments which activate the latently viral-infected cells, such as latently HIV-infected cells. In one embodiment, the sheath, shell, polymeric shell, cover, casing, encoating, or jacket is dextran, a polyglucose, polysaccharide with its extensive history of clinical use in millions of patients

Also disclosed is a method for potentiating, sensitizing, and/or amplifying adjuvant pharmaceuticals to make the cells of a patient more sensitive to the cytopathic activity of the adjuvant pharmaceuticals which are being used in conjunction with HAART for activating proviral HIV infection by administering to the patient a composition comprising a colloidal solution having a core of at least a biologically acceptable fixed copper compound or a biologically acceptable insoluble iron compound or mixtures thereof wherein said core is encapsulated, encoated, adsorbed, complexed or bound in at least one of a sheath, a shell, a polymeric shell, a cover, a casing, an encoating, a jacket or combination thereof, and a pharmaceutically acceptable carrier. The sheath, shell, polymeric shell, cover, casing, encoating, jacket or combination thereof prevents immediate chemical interaction of the core with the surrounding environment. The method also includes the administration of at least one proviral stimulant with conventional ART/HAART treatments which activate the latently viral-infected cells, such as latently HIV-infected cells. The patient is monitored regularly to determine the level and/or presence of the HIV infection. The composition may be re-administered at intervals determined to be medically necessary by the physician, based on the results of the monitoring.

The present invention is advantageously a safe and effective composition which employs bio-compatible materials which feed every cell in the body to selectively amplify and potentiate adjuvant pharmaceuticals, such as proviral stimulants, so that latently infected cells are currently kicked and, with the invention, killed without harming normal cells. Therefore, the present disclosure is generally directed to a method and composition to suppress, kill and “get rid of it” as recommended necessary by Fauci.

### DETAILED DESCRIPTION

Without limitation, these and other objects, features, and advantages of the present invention, will become apparent to those with skill in the art after review of the following detailed description of the disclosed embodiments. While not being limited, held or bound to any particular theory or mechanism of action, the applicant discloses the detailed description of the invention.

This disclosure is directed to a method for potentiating, sensitizing, and/or amplifying at least one adjuvant pharmaceutical targeting at least one latent viral infection in a patient comprising: forming a composition including a colloidal solution having a core of at least a biologically acceptable fixed copper compound or a biologically acceptable insoluble iron compound or mixtures thereof wherein said core is encapsulated, encoated, adsorbed, complexed or bound in at least one of a sheath, a shell, a polymeric shell, a cover, a casing, an encoating, a jacket or combination thereof, and a pharmaceutically acceptable carrier; said sheath, shell, polymeric shell, cover, casing, encoating, jacket or combination thereof preventing immediate chemical interaction of the core with the surrounding environment; and administering the composition to the patient to potentiate, sensitize, and/or amplify at least one adjuvant targeting at least one latent viral infection in the patient. In one embodiment, the composition is administered to potentiate an adjuvant pharmaceutical targeting at least one latent viral infection such as those which are currently being investigated for use with anti-HIV drugs/antiretrovirals HAART(See United States Department of Veterans Affairs, Treatment Decisions for HIV). Adjuvants pharmaceuticals are agents that aid or increase the action of the principal drug or that affect the absorption, mechanism of action, metabolism, or excretion of the primary drug in such a way as to enhance its effects.

This disclosure is also directed to a chemical formulation for potentiating, sensitizing, and/or amplifying adjuvants pharmaceuticals used in conjunction with antiretroviral therapy comprising a composition including a colloidal solution having a core of at least a biologically acceptable fixed copper compound or a biologically acceptable insoluble iron compound or mixtures thereof wherein said core is encapsulated, encoated, adsorbed, complexed or bound in at least one of a sheath, a shell, a polymeric shell, a cover, a casing, an encoating, a jacket or combination thereof, and a pharmaceutically acceptable carrier; said sheath, shell, polymeric shell, cover, casing, encoating, jacket or combination thereof preventing immediate chemical interaction of the core with the surrounding environment. This is known as a core/shell configuration in the art and is also disclosed in U.S. Pat. No. 7,449,196 to Sabin, the contents of which are expressly incorporated herein by reference. The composition is administered to patients taking at least one proviral stimulant with or without conventional ART/HAART treatments which activate the latently viral-infected cells, such as latently HIV-infected cells. In one embodiment, the proviral stimulants are HIV proviral stimulant selected from the group consisting of panobinostat, givinostat, belinostat, vorinostat, valproic acid, disulfram, certain interleukins, HDAC Inhibitors and combinations thereof.

Also disclosed is a method for potentiating adjuvant pharmaceuticals to make the cells of a patient more sensitive to the cytopathic activity of the adjuvant pharmaceuticals which are being used in conjunction with HAART for activating proviral HIV infection by administering to the patient a composition comprising a colloidal solution having a core of at least a biologically acceptable fixed copper compound or a biologically acceptable insoluble iron compound or mixtures thereof wherein said core is encapsulated, encoated, adsorbed, complexed or bound in at least one of a sheath, a shell, a polymeric shell, a cover, a casing, an encoating, a jacket or combination thereof, and a pharmaceutically acceptable carrier; said sheath, shell, polymeric shell, cover, casing, encoating, jacket or combination thereof preventing immediate chemical interaction of the core with the surrounding environment. The method also includes the administration of at least one proviral stimulant with conventional ART/HAART treatments which activate the latently viral-infected cells, such as latently HIV-infected cells. The patient is monitored regularly to determine the level and/or presence of the HIV infection. The composition may be re-administered at intervals determined to be medically necessary by the physician, based on the results of the monitoring.

The composition is comprised of, at least, nanoparticles of a fixed copper compound core, or an insoluble iron compound core, or a combination of the two formed separately. The nanoparticles of an fixed copper compound core are also referred to herein as “copper dextran”. The nanoparticles of an insoluble iron compound core are also referred to herein as “iron dextran”. It is understood that the use of “dextran” in “copper dextran” and “iron dextran” includes other encapsulating material as further disclosed herein and is not strictly limited to dextran per se. The term “fixed” with respect to copper compounds herein is defined herein based on the compound's insolubility or highly insolubility in water. The term “insoluble” with respect to iron compounds refers herein to a compound which is insoluble or highly insoluble in water. These cores may be encapsulated, coated, adsorbed, complexed, or the like, with a protective sheath or jacket which also functions to target latent viral infections. This sheath or jacket may be any combination of materials, such as a glucose or liposome, and, optionally, the resulting glucose encapsulated core may be coated with liposomes. In another embodiment, the core may be encapsulated with dextran alone or any glucose or combination of sugar-based substances. Alternatively, a liposome encapsulated core may then be coated with an outer dextran sheath.

Suitable copper compounds for use as the core are any biologically acceptable copper compounds which include, but are not limited to, any fixed coppers including, cupric hydroxide, copper oxide, copper oxychloride, cupric carbonate basic, copper sulfate, copper sulfate basic, cuprous oxide, cupric hydroxide-iron hydroxide, copper-iron oxide, cupric citrate, cupric glycinate, cupric gluconate, cupric phosphate, cuprobam, cupric salicylite, indigo copper, cupro-cupric sulfate, cuprous sulfate, cuprous sulfate hemihydrite, any of the natural copper containing minerals such as cupric sulfate basic, the minerals brochantite, langite, malachite, azurite, cheesylite, cornetite, dihydyrite, libethenite, phosphorochalcite, pseudolibethenite, pseudomalachite, tagilite, antlerite, covellite, marshite, cuprite, chalcocite, Rogojski's salt, brochantite, hydrocyanite, chalcanthtite, and the like, or any copper minerals occurring in nature such as nantokite or dolerophane and so on. See also, for examples of copper compounds, Merck's Manual 13.sup.th ed., Merck & Co. 2001, and Hawley's Condensed Chemical Dictionary 14.sup.th ed., John Wiley & Sons, Inc. 2001. Copper hydroxide, an insoluble, fixed copper, is a preferred compound to form the core. In another embodiment, the core may also be composed of cupric hydroxide-iron hydroxide to provide a synergistic effect, which enhances the cellular toxicity of both the copper and iron. In one embodiment, any biocompatible form of copper compound that can cause catalysis of free-radical reactions in biological systems may be used as a core metal for the disclosed composition. A biologically acceptable copper compound as defined herein is a copper compound, which may be used with and within a biological system with little or no detrimental effect, i.e. it does not appreciably alter or appreciably affect in any adverse way, the biological system into which it is introduced.

In a further embodiment, a combination of copper oxide, copper hydroxide-iron hydroxide or another of the fixed (insoluble or highly insoluble) coppers and insoluble iron, may be used as a core to provide synergistic effects of the combination. Any biocompatible iron compound may be used in conjunction with the copper core, including without limitation, for example, Fe³⁺, and its salts, iron hydroxide, iron oxyhydroxide, iron oxide, and the like, to iron load the biological environment, including iron-saturated human holotransferrin.

The nanoparticles of the disclosed Composition preferably can be encapsulated, surrounded, complexed, or adsorbed by, and bound to, at least one sheath or coat that is preferably composed of a sugar substance, such as a glucose, a saccharide, a polysaccharide e.g. starch, cellulose, dextrans, alginides, chitosan, pectin, hyaluronic acid, pullulan (a bacterial polysaccharide), dextran, carboxyalkyl dextran, carboxyalkyl cellulose and the like. These dextrans can include, for example, those disclosed by Mehvar, supra (2000); and Recent Trends in the Use of Polysaccharides for Improve Delivery of Therapeutic Agents: Pharmacokinetic and Pharmacodynamic Perspectives, Curr. Pharm. Biotech. 4:283-302 (2003), and liposomes coated with dextran as disclosed by Moghimi, et al., Long-Circulating and Target-Specific Nanoparticles: Theory to Practice, Pharm. Rev., 53(2):283-318 (2001) both of which are incorporated herein in their entirety. The sheath encoats, or encapsulates, the disclosed Composition's core and prevents chemical interaction of the core with the surrounding environment, blocking the degradation of the core and the emanation of the copper and/or iron from the copper compound, and/or the copper-iron compound from the core. The thickness of the sheath may be varied, if desired, by those skilled in the art. Because the sheath is composed primarily of a substance that is not necessarily recognized by the body as foreign matter, the body is less likely to develop a resistance to the Composition. In one embodiment, the sheath can be composed of dextran, also known as macrose, a high molecular weight polysaccharide. Dextran is an ideal candidate for use as a sheath because it is often administered to mammals as a blood plasma substitute or expander, is generally not rejected by the mammalian system, and can remain in the plasma for an extended period of time. Other biocompatible materials for the formation of a polymeric shell, sheath, or jacket can include proteins, polypeptides, oligopeptides, polynucleotides, polysacchrides, lipids and so on. Additional sheath materials include, for example, those of U.S. Pat. Nos. 6,096,331; and 6,506,405, incorporated herein in their entirety. Alternatively, combinations of two or more of the above named materials may be used to form the sheath.

In another embodiment, the disclosed Composition can be sheathed or encapsulated with a liposome coat. This liposome coat may be the sole sheath encapsulating the core, or may be a second coat over one, or a combination, of the above named materials. PEG liposome polymer coatings have been shown to reduce phagocytic system uptake and provide long residence time according to research by the Alza Corporation, Delivery Times, Issues and Opportunities, Vol 2 (1), incorporated herein in its entirety. Residence time in the plasma can be extended to periods of at least several days to weeks after IV injection without releasing the encapsulated drug, which would lower the administration frequency of the drug. See, e.g., U.S. Pat. No. 6,465,008; U.S. Pat. Pub. U.S. 2002/017271181; U.S. Pat. Pub. U.S. 2001/005118381; each of which is incorporated herein in its entirety.

Alternatively, the core may be transported to cell-specific sites with the use of targeting agents or markers which may target latent viral infections. Any targeting agent or marker which can medicinally utilized within a biological system may be employed to actively transport the core to the specific site (See, for example, R. C. Juliano, Targeted Drug Delivery, Handbook of Experimental Pharmacology, Vol. 100, Ed. Born, G. V. R. et al., Springer Verlag). These targeting agents or markers may be used instead of, or in conjunction with, at least one sheath encapsulating the core.

The nanoparticle size of the entire disclosed Composition may be approximately 1 nm to approximately 10,000 nm. In a more preferred embodiment, the particle size may be approximately 15 nm to approximately 500 nm. A most preferred embodiment for particle size is approximately 20 nm to approximately 200 nm.

Empty liposomes, which are devoid of drugs, may be co-administered or administered before, during, or after the Composition itself to the patient, to function as a decoy, placebo carrier, or redistribution agent with respect to the phagocytic system and allow the Composition to remain in the plasma for an extended period of time. The empty liposome decoys, or placebo carriers, occupy the phagocytic system and also redistribute the disclosed composition away from clearance by cells in the liver and in the spleen and thus concentrate the disclosed composition in the plasma for an extended period of time. Biocompatible materials used for polymeric shells may also be employed as decoys, alone or in combination with liposomes.

In one embodiment, the disclosed copper-core composition plus iron dextran plus empty liposomes may be added to the total parenteral nutrition (“TPN”) for the patient. The disclosed composition includes essential trace elements of copper, and may include iron, as well as glucose, and/or liposomes, which are fats, to contribute to the patient's bodily requirements. Thus the Composition also provides an important contribution to the total parenteral nutrition of the patient.

Without being limited, held, or bound to any particular theory or mechanism of action, it is believed that the Composition, the redistribution agents, i.e., iron dextran with or without empty liposomes, enters the system, traffics throughout the body as an inert entity, and is removed from the plasma by the phagocytic system. The Composition can remain in the mammal's plasma for a period of many days, depending on the dosage levels, when used with a redistribution agent or placebo carrier. (It is known that iron-dextran can remain in the plasma for weeks, especially when doses are administered above the clearance rate of the phagocyte system. The processing of the iron dextran by the phagocytic system is rate limited to a daily maximum amount, leaving the balance for future use.) The sheath may not be immediately recognized as foreign matter by the phagocytic system because it is a sugar-based substance and is not rejected by the mammalian system, allowing the Composition to remain in circulation of the mammal for a longer period than most therapeutics, making it more likely to come into contact with target cells and providing more efficacy with fewer doses than traditional agents. The Composition circulates, via any biological pathway, throughout the body and may contact any cell type. For the most part, the phagocytic system takes up the Composition. Normal, healthy cells generally have very little interaction with the Composition. The Composition that is taken up by the phagocytic system is processed, to a large degree, through the liver in hepatocytes that store glucose, iron, and copper and are later released through their appropriate protein carriers to feed and nurture cells of the body. Since sugars, copper, and iron are bodily requirements, well known to the phagocytic system, the phagocytic system is able to process, transport, store, or eliminate them with little toxicity, while the Composition potentiates, sensitizes, and/or amplifies at least one adjuvant pharmaceutical targeting at least one latent viral infection in a patient it simultaneously feeds and nourishes cells in the body.

The administration of iron compositions and/or iron dextran compositions may be combined with the disclosed copper-core Composition to provide synergistic reactions between the copper and iron. The synergy between copper and iron is known in the art, and has been described in the literature, see, for example, U.S. Pat. No. 5,202,353, incorporated herein in its entirety, which discloses use of the synergistic affects of copper compositions and iron compositions for use as fungicides and bactericides. The iron compositions and/or iron dextran compositions may also be administered to redistribute the disclosed Composition and allow the Composition a longer residence time in the patient's plasma. Far higher dosages of iron dextran may be employed, than that of elemental iron salts, for a greater cytotoxicity, and a protracted residence plasma time. The greater the iron level, the greater the synergistic cytotoxicity of the Composition. Because it is well known in the art that the phagocytic system removes the smaller particles from the plasma circulation first, the combination of the iron dextran with a smaller diameter than the Composition allows a protracted plasma residence time. The diameters of the iron dextran and the core of the disclosed Composition may be varied to manipulate the plasma time of these particles as desired. In one embodiment, the iron dextran can be administered above the clearance level of the phagocyte system, which can serve as a decoy, placebo carrier, or redistribution agent to allow the Composition to remain in the plasma for an extended period of time. (See, Henderson & Hillman, Characteristics of Iron Dextran Utilization in Man, Blood, 34(3):357-375(1969)). This use of iron dextran at a dose above the rate of clearance of the phagocyte system, to allow the disclosed Composition to remain in the plasma for an extended period of time, is known in the art as a redistribution (away from the liver and spleen to the plasma). Generally, smaller doses of iron dextran (50-500 mg) are cleared within approximately 3 days, larger doses of iron dextran (>500 mg), however, are cleared at a constant rate of 10-20 mg/hr and are typically associated with increased plasma concentration of iron dextran for as long as 3 weeks. Other agents which may serve as decoys for the phagocytic system to redistribute the disclosed Composition to the plasma include, without limitation, pullulan, dextran sulfate, empty liposomes, and those taught by U.S. Pat. Nos. 6,506,405, and 6,096,331 incorporated herein in their entirety.

Since the disclosed composition, iron dextran, and empty liposomes are all formed of biocompatible materials, all may be administered over an extended period of time as compared to other chemotherapeutic agents. The effective dose or effective amount can vary subject to the evaluation of the those of skill in the art in relation to the particular type of viral infection, the regimen of administration, the body weight of the subject, the aggressiveness of the viral infection and the degree in which the subject has been negatively affected by prior therapy.

The disclosed Composition can be administered to a patient as a pharmaceutical composition in combination with a pharmaceutical carrier. A pharmaceutical carrier can be any compatible, non-toxic substance suitable for delivery of the Composition to the patient that is medically acceptable. Sterile water, alcohol, fats, waxes, and inert solids may be included in the carrier. Pharmaceutically accepted adjuvants (buffering agents, dispersing agent) may also be incorporated into the pharmaceutical compound. In one embodiment, the Composition may be combined with sterile water, or deinozed water and free dextran, dextran free of drug, to form a sterile colloidal suspension.

The disclosed Composition may be administered to a patient in a variety of ways, such as oral, intravenous, subcutaneous, intraperitoneal, intrathecal, intramuscular, intracranial, inhalational, topical, transdermal, suppository (rectal), pessary (vaginal) or an implantable polymer disclosed composition saturated depot or wafer, such as, for example, a Giladel wafer®. Preferably, the pharmaceutical compound may be administered parenterally, e.g., subcutaneously, intramuscularly or intravenously. Thus, the disclosed Composition may include a solution dissolved in an acceptable carrier, preferably an aqueous carrier, for parenteral administration. A variety of aqueous carriers can be used, e.g., water, buffered water, 0.4% saline, 0.3% glycine and the like. These solutions are sterile and generally free of particulate matter. These compounds may be sterilized by conventional, well-known sterilization techniques. The Composition may contain pharmaceutically acceptable auxiliary substances as required to approximate physiological conditions such as pH adjusting and buffering agents, and if necessary for sensitive patients, toxicity adjusting agents and the like, for example sodium acetate, sodium chloride, potassium chloride, calcium chloride, sodium lactate, etc. The concentration of the disclosed Composition in these formulations can vary widely, e.g., from less than about 0.1 mg to about 5 mg, ranging to as much as 10 mg or 15 mg or more of the equivalent of elemental copper derived from the Composition per ml of carrier. The preferred concentration of the disclosed Composition is approximately 5 mg of the equivalent of elemental copper derived from the Composition per ml of carrier, and will be selected primarily based on fluid volumes, viscosities, etc., in accordance with the particular mode of administration selected. The preferred pH range for use with the disclosed Composition is between approximately 7 and approximately 8.5, and the more preferred pH range is between approximately 7.5 and approximately 8.0.

Actual methods for preparing parenterally administerable compounds and adjustments necessary for administration to patients, typically mammals, will be known or apparent to those skilled in the art and are described in more detail in, for example, Remington's Pharmaceutical Science: The Science and Practice of Pharmacy, 20.sup.th Ed., Lippincott, Williams & Wilkins; (2000), which is incorporated herein by reference.

In one embodiment, the colloidal solution includes a core of a biologically acceptable insoluble iron compound encapsulated, encoated, adsorbed, complexed or bound to dextran. A biologically acceptable iron compound as defined herein is an iron compound, which may be used with and within a biological system with little or no detrimental effect, i.e. it does not appreciably alter or appreciably affect in any adverse way, the biological system into which it is introduced. In one embodiment, the insoluble iron compound can be selected from the group consisting essentially of iron oxide, iron hydroxide, and iron oxyhydroxide. Iron dextran is the perfect HIV antiviral. It traffics all throughout the body and crosses the blood/brain barrier (see Neuwelt, E.A., “Imaging of iron oxide nanoparticles by MR and light microscopy in patients with malignant brain tumours', Neuropathal Appl Neurobiol. 2004 October; 30(5); 456-71, which discloses that dextran encapsulated/coated nanoparticles cross the blood/brain barrier which is a sanctuary site for HIV virus). The only problem with iron dextran as an anti-HIV pharmaceutical is that it has little or no anti-viral activity and may promote opportunistic infections. The advantages of iron dextran as a potentiator to the adjuvant pharmaceuticals currently being investigated with HAART are numerous. It is understood that any discuss herein relating of HIV-infected cells may be equally applied to viral-infected cells generally

First, dextran is a polyglucose. It is well known and established to the scientific and medical community as a plasma volume expander and as the polymeric shell of iron dextran with several hundreds of millions safe administrations. Moreover, according to the FDA approved package insert for INFED®, dextran is metabolized or excreted, iron is stored, and the PK is known. There have been at least 100 million administrations of iron dextran throughout the world for about 59 years.

Dextran is also cheap, generic and best of all, a polyglucose, with the latently infected CD-1 memory cells being lymphocytes, phagocytic, with a predilection for glucose. Moreover, macrophages and monocytes thought to be latently HIV infected also have a predilection for glucose, avidity for glucose, and are phagocytic.

Glucose increases the metabolic rate of phagocytic cells so that it may help in activating these cells to synergize cytotoxicly, more kick, with VOR or the like, the kicker. The iron, which generates ROS, may also add more kick, promote cell killing.

Perhaps one of the most critical and important part for this invention, iron dextran can load the latently HIV-infected resting memory CD4-positive T cells and other latently HIV-infected cells such as macrophages and monocytes which are phagocytic with a predilection for glucose with iron without affecting normal cells and causing much toxicity. At this point with the iron dextran loading the latently HIV-infected resting memory CD4-positive T cells and other latently HIV-infected cells for weeks, when the HDAC inhibitor or other proviral infected cell activator is administrated, such as vorinostat, there may be enough ROS generation from the iron derived from the iron dextran to cause cell death. (See Matalon, “Histone deacetylate inhibitor for purging HIV-1 from the latent reservoir, Mol Med. 2011 May-June; 17 (5-6); 466-72. See Rasmussen, “Comparison of HDAC inhibitors in clinical development: Effect on HIV production in latently infected cells and T-cell activation”, Hum Vaccin Immunother, 2013 Jan. 31; 9(5)) The vorinostat or other HDAC inhibitors or other proviral activators spare normal cells which are not infected, therefore not activated, and are therefore thought to be more robust. It is thought that latently infected cells are more fragile than normal cells and that the combination of iron loading and vorinostat or other HDAC inhibitors or other latently infected virus activators will push the cell over the edge and kill it, especially when the cell is activated to undergo transcription and produce virus.

Applicant has administered his copper dextran to macrophages and monocytes, some of which are thought to be latently infected with HIV. Applicant has achieved high activity in these cells which prove that the dextran sheath is taken up by macrophages and monocytes.

In a preferred embodiment, the insoluble iron core may include iron hydroxide, iron-oxyhydroxide, or iron oxide or other insoluble iron compounds with a shell of dextran or shells as disclosed in U.S. Pat. No. 6,096,331 of Desai (page 9, paragraphs 7-9) and (page 10, paragraphs 1-2). This insoluble iron core is a biological, compatible material that does not significantly change or affect in any detrimental manner, the living system into which it is utilized. U.S. Pat. No. 5,624,668 of Lawrence, for Iron Dextran Formulations, discloses how to manufacture such iron dextran formulation. Lawrence also discloses information about iron dextran formulations in his following paper: “Development and Comparison of Iron Dextran Products, Journal of Pharmaceutical Science and Technology, Vol. 52, No. 5, September-October 1998, pgs 190-197.

Iron dextran, although it has perfect pharmacokinetics for latent HIV infection, may not be effective enough to generate enough ROS (reactive oxygen species) with vorinostat for cell kill. Therefore, the addition of fixed copper compounds and copper iron compound which are encapsulated in dextran, like iron dextran may be used in the composition as disclosed in U.S. Pat. No. 7,449,196 of Sabin. This dextran encapsulated copper compound has the ability to generate abundant ROS, so that it kills tumor cells and the cytopathic effect is potentiated with an adjuvant of iron dextran. (See FIG. 1 in Sabin U.S. Pat. No. 7,449,196) The addition of iron dextran to the applicant's copper drug increased ROS production and cytotoxicity in ten major tumor cell lines so that less copper was needed for an IC50 (see Table 1 in Sabin).

The encapsulated fixed core copper compounds have been administered together in the same syringe with iron dextran to potentiate the cytotoxic effect. These fixed core copper compounds have also been tested against HIV infection in vitro and have good activity. (See U.S. Patent Application Publication 20060147512 of Sabin) Moreover U.S. Patent Application Publication 20060147512 of Sabin, demonstrates that the dextran encapsulation on the copper compounds enters, is taken in, internalized by the HIV infected Peripheral Blood Mononuclear Cells (PBMCs) [y1], so that the iron dextran with the same dextran shell will also be internalized by HIV infected cells.

The encapsulated fixed copper dextran may be added to the iron dextran so that it will increase the reactive oxygen species and deliver all the cytotoxicity desired. The finesse object of the invention is to iron and copper load (loading dose is defined as a large initial dose of a substance or series of such doses given to rapidly achieve a therapeutic concentration in the body) the latently HIV infected cell, to less than where it is cytotoxic so that, at this sweet spot, when the proviral activator is taken in the cell and activated, there will be cell killing—to put the kill in “kick and kill” “Sweet spot” is defined as where the internal concentrations of copper and iron are elevated and increased without cell kill. Further increases in the internal concentration of copper and iron could kill the cells so that it is unworkable to kill one million cells to get at killing one provirally infected cell. With current “kick and kill”, there is no “kill”. Applicant feels that there is an enormous amount of work being done to develop more active HIV proviral activators, but if these better, superior activators do not kill the cell, we will be worse off than we were before because they will be activating possibly a broader population of latently infected cells. Combination of iron dextran and copper dextran have been administered in the same syringe to dozens of laboratory BALB/C mice by I.V. and I. P. uneventfully without toxicity. Moreover, the antiviral activity and the cytotoxicity increase markedly from administering copper alone or iron alone.

It is an object of the present invention to load latently infected HIV cells throughout the body with iron dextran and a small amount of copper dextran which will act as an adjuvant to add to the iron dextran which is an adjuvant to the HIV proviral stimulants, such as vorinostat, which are adjuvants to HAART. At the end of the day, the applicant's invention is called SEEK, LOAD, KICK and KILL. The applicant's invention adds SEEK and LOAD to KICK and KILL so that the latently infected cells will be killed with modest acceptable toxicity. By adding the applicant's copper dextrans or copper iron dextrans to the iron dextran, the skilled artisan can derive all the ROS and antiviral/cytotoxic activity he desires. The range of copper derived from copper dextran to add to the iron derived from iron dextran will be from 0 to 50 ug/ml of blood. A more preferred range is 0 to 10 ug/ml. A further more preferred range is 0 to 3 ug/ml and a further more preferred range is 0 to 1 ug/ml. (See U.S. Patent Application Publication 20060147512 of Sabin) Those of skill in the art can easily calculate patient weight, blood volume and the amount of copper dextran to be administered to achieve the levels desired. Moreover, copper is not stored in the body; the body containing about 150 milligrams of copper at any one time and any excess copper is excreted through the biliary system. Most of the world is iron deficient and iron is stored in the body in the liver, generally, about 4-5 grams.

Applicant's compounds, iron dextran and his dextran encapsulated copper compounds, are to be administrated by injection, IV IP, etc. (See Administration of Intravenous Iron Dextran, sickle.bwh.harvard.edu) Applicant's dextran encapsulated copper compounds and iron dextran may be combined in the same syringe so that the medical team can calculate exactly how long the compounds will remain in the plasma, traffic all throughout the body in the plasma, hit or expose most or all of the HIV sanctuary sites, including but not limited to the lymphatic system, GALT, marrow, CNS system, across and crossing the blood/brain barrier. By example, U.S. Pat. No. 5,624,668 of Lawrence discloses in Example 3, Table 3, that the sole current FDA approved iron dextran, INFED®, by way of a single 100 mg intravenous dose has a plasma residence time, half life of 34.2 hours so that the applicant's copper dextran and iron dextran compounds will hit essentially all the sites of HIV infection. This half life is many times extended by doses of 500 mg, 1000 mg, 2000 mg, 3000 mg which have been safely administered to millions of people and hundreds of millions of livestock throughout the world. The medical team treating HIV infection/AIDS will decide exactly how long they want the agents to hit the latently infected cells, sanctuary sites and all the other HIV infected cells. It could be two weeks+ by one single TDI administration. The HIV virus is well known in the art to be very persistent, stubborn, sneaky, so that applicant's composition will be there 24/7 for weeks; the copper being excreted, glucose being metabolized or excreted and the iron being stored with the bulk of people infected with HIV in Africa being deficient in iron, copper, glucose/micronutrients, so that they will get treated and fed at the same time.

In one embodiment, in order to promote the ingestion of the applicant's compounds with a polyglucose sheath, insulin may also be utilized as it promotes the uptake of glucose into cells. (See IPT Therapy: Insulin Potentiating Therapy)

The composition of applicant's invention may be administered separately or together in the same syringe/I.V. Bag. For Example, iron dextran may be administered on Day One, 24 hours later, the encapsulated copper compound may be administered via I.V., 2 hours or more or less later, the HIV proviral stimulants such as panobinostat, givinostat, belinostat, vorinostat, valproic acid, or others may be administered alone or in combination.

In one embodiment, the FDA-approved iron dextran, INFED®, with a size of about 90 nm (tested with Laser Light Scattering) may be administered to a patient with a proviral infection or a patient infected with HIV, or thought to be infected with HIV, taking or not taking HAART/ART in the same I.V. bag/syringe with the encapsulated copper compound/copper dextran with a size of about 90 nm on Day One. 8-24 Hrs later the HIV proviral stimulant such as, for example, Disulfram, and HDAC Inhibitors such as Vorinostat, panobinostat, or other HDAC inhibitors or any other proviral stimulators such as biological proviral stimulators, may be administered alone or in combination.

In the foregoing description, certain terms and visual depictions are used to illustrate the preferred embodiment. However, no unnecessary limitations are to be construed by the terms used or illustrations depicted, beyond what is shown in the prior art, since the terms and illustrations are exemplary only, and are not meant to limit the scope of the present invention.

It is further known that other modifications may be made to the present invention, without departing the scope of the invention, as noted in the appended Claims.

I claim:

1. A method for potentiating, sensitizing, and/or amplifying at least one adjuvant pharmaceutical targeting at least one latent viral infection in a patient comprising:
forming a composition including a colloidal solution having a core of at least a biologically acceptable fixed copper compound or a biologically acceptable insoluble iron compound or mixtures thereof, wherein said core is encapsulated, encoated, adsorbed, complexed or bound in at least one of a sheath, a shell, a polymeric shell, a cover, a casing, an encoating, a jacket or combination thereof, and a pharmaceutically acceptable carrier;
said sheath, shell, polymeric shell, cover, casing, encoating, jacket or combination thereof preventing immediate chemical interaction of said core with the surrounding environment; and
administering the composition to the patient to potentiate, sensitize, and/or amplify the at least one adjuvant targeting the at least one latent viral infection in the patient.

2. The method of claim 1, wherein adjuvant pharmaceutical is an HIV proviral stimulants.

3. The method of claim 2, wherein said HIV proviral stimulant is selected from the group consisting of panobinostat, givinostat, belinostat, vorinostat, valproic acid, disulfram, interleukins, HDAC Inhibitors, and combinations thereof.

4. The method of claim 1, wherein said at least one latent viral infection is a latent HIV-infection.

5. The method of claim 1, wherein the composition includes a mixture of separately formed cores of biologically acceptable fixed copper compound and cores of biologically acceptable insoluble iron compound.

6. The method of claim 1, wherein said step of administering said composition includes separately administering said colloidal solution having a core of at least a biologically acceptable fixed copper compound and then administering said colloidal solution having a core of at least a biologically acceptable insoluble iron compound.

7. The method of claim 1, wherein said biologically acceptable fixed copper compound is selected from the group consisting of cupric hydroxide, copper oxide, copper oxychloride, cupric carbonate basic, copper sulfate basic, cuprous oxide, cupric hydroxide-iron hydroxide, copper-iron oxide, cupric citrate, cupric phosphate, cuprobam, indigo copper, minerals brochantite, langite, malachite, cornetite, libethenite, pseudolibethenite, pseudo-malachite, antlerite, covellite, marshite, cuprite, chalcocite, Rogojski's salt, brochantite, hydrocyanite, nantokite and dolerophane,

8. The method of claim 1, further comprising parenterally administering the composition to the patient.

9. The method of claim 1, further comprising orally administering the composition to the patient.

10. The method of claim 1, further comprising transdermally administering the composition to the patient.

11. The method of claim 1, wherein the composition is administered for the total parenteral nutrition of a patient.

12. The method of claim 1, wherein the composition is administered with insulin potentiation therapy of a patient.

13. The method of claim 1 wherein said sheath, shell, polymeric shell, cover, casing, encoating, jacket or combination thereof includes a substance selected from the group consisting of glucose, a saccharide, a polysaccharide, a carbohydrate, a protein, a dextran, a fat, a liposome, derivatives thereof or combinations thereof.

14. The method of claim 1 wherein said core comprises nanoparticles covered by sheath material.

15. The method of claim 1 wherein a biocompatible iron compound is used in conjunction with the core of at least a biologically acceptable copper compound.

16. The method of claim 15 wherein said biocompatible iron compound is selected from the group consisting of iron hydroxide, iron oxyhydroxide and iron oxide.

17. The method of claim 1 wherein said biologically acceptable insoluble iron compound is selected from the group consisting of iron hydroxide, iron oxyhydroxide and iron oxide.

18. A chemical formulation for potentiating, sensitizing, and/or amplifying adjuvants pharmaceuticals used in conjunction with antiretroviral therapy comprising
a composition including a colloidal solution having a core of at least a biologically acceptable fixed copper compound or a biologically acceptable insoluble iron compound or mixtures thereof wherein said core is encapsulated, encoated, adsorbed, complexed or bound in at least one of a sheath, a shell, a polymeric shell, a cover, a casing, an encoating, a jacket or combination thereof, and a pharmaceutically acceptable carrier; said sheath, shell, polymeric shell, cover, casing, encoating, jacket or combination thereof preventing immediate chemical interaction of the core with the surrounding environment; and
at least one proviral stimulant.

19. The chemical formulation of claim 18, wherein said proviral stimulant is an HIV proviral stimulants.

20. The chemical formulation of claim 19, wherein said HIV proviral stimulant is selected from the group consisting of panobinostat, givinostat, belinostat, vorinostat, valproic acid, disulfram, interleukins, HDAC Inhibitors, and combinations thereof.

21. The chemical formulation of claim 18, wherein the composition includes a mixture of separately formed cores of biologically acceptable fixed copper compound and cores of biologically acceptable insoluble iron compound.

22. The chemical formulation of claim 18, wherein said biologically acceptable fixed copper compound is selected from the group consisting of cupric hydroxide, copper oxide, copper oxychloride, cupric carbonate basic, copper sulfate basic, cuprous oxide, cupric hydroxide-iron hydroxide, copper-iron oxide, cupric citrate, cupric phosphate, cuprobam, indigo copper, minerals brochantite, langite, malachite, cornetite, libethenite, pseudolibethenite, pseudomalachite, antlerite, covellite, marshite, cuprite, chalcocite, Rogoj ski's salt, brochantite, hydrocyanite, nantokite and dolerophane,

23. The chemical formulation of claim 18, wherein the sheath, shell, polymeric shell, cover, casing, encoating, jacket or combination thereof includes a substance selected from the group consisting of glucose, a saccharide, a polysaccharide, a carbohydrate, a protein, a dextran, a fat, a liposome, derivatives thereof or combinations thereof.

24. The chemical formulation of claim 18 in which the core comprises nanoparticles covered by sheath material.

25. The chemical formulation of claim 18 wherein a biocompatible iron compound is used in conjunction with the core of at least a biologically acceptable copper compound.

26. The chemical formulation of claim 25 in wherein said biocompatible iron compound is selected from the group consisting of iron hydroxide, iron oxyhydroxide and iron oxide

27. The chemical formulation of claim 18 wherein said biologically acceptable insoluble iron compound is selected from the group consisting of iron hydroxide, iron oxyhydroxide and iron oxide.

28. A method for potentiating, sensitizing, and/or amplifying adjuvant pharmaceuticals to make the cells of a patient undergoing highly active antiretroviral therapy more sensitive to the cytopathic activity of the adjuvant pharmaceuticals which are being used in conjunction with highly active antiretroviral therapy for activating proviral HIV infection comprising:
administering to said patent undergoing highly active antiretroviral therapy a composition comprising a colloidal solution having a core of at least a biologically acceptable fixed copper compound or a biologically acceptable insoluble iron compound or mixtures thereof wherein said core is encapsulated, encoated, adsorbed, complexed or bound in at least one of a sheath, a shell, a polymeric shell, a cover, a casing, an encoating, a jacket or combination thereof, and a pharmaceutically acceptable carrier; said sheath, shell, polymeric shell, cover, casing, encoating, jacket or combination thereof preventing immediate chemical interaction of the core with the surrounding environment;
administering at least one adjuvant pharmaceutical to said patient; and
administering at least one antiretroviral drug to said patient.

29. The method of claim 28, wherein adjuvant pharmaceutical is an HIV proviral stimulants.

30. The method of claim 29, wherein said HIV proviral stimulant is selected from the group consisting of panobinostat, givinostat, belinostat, vorinostat, valproic acid, disulfram, interleukins, HDAC Inhibitors, and combinations thereof.

31. The method of claim 28, wherein the composition includes a mixture of separately formed cores of biologically acceptable fixed copper compound and cores of biologically acceptable insoluble iron compound.

32. The method of claim 28, wherein said step of administering said composition to said patient includes separately administering said colloidal solution having a core of at least a biologically acceptable fixed copper compound and then administering said colloidal solution having a core of at least a biologically acceptable insoluble iron compound.

33. The method of claim 28, wherein said biologically acceptable fixed copper compound is selected from the group consisting of cupric hydroxide, copper oxide, copper oxychloride, cupric carbonate basic, copper sulfate basic, cuprous oxide, cupric hydroxide-iron hydroxide, copper-iron oxide, cupric citrate, cupric phosphate, cuprobam, indigo copper, minerals brochantite, langite, malachite, cornetite, libethenite, pseudolibethenite, pseudomalachite, antlerite, covellite, marshite, cuprite, chalcocite, Rogojski's salt, brochantite, hydrocyanite, nantokite and dolerophane,

34. The method of claim 28, further comprising parenterally administering the composition to the patient.

35. The method of claim 28, further comprising orally administering the composition to the patient.

36. The method of claim 28, further comprising transdermally administering the composition to the patient.

37. The method of claim 28, wherein the composition is administered for the total parenteral nutrition of a patient.

38. The method of claim 28, wherein the composition is administered with insulin potentiation therapy of a patient.

39. The method of claim 28 wherein said sheath, shell, polymeric shell, cover, casing, encoating, jacket or combination thereof includes a substance selected from the group consisting of glucose, a saccharide, a polysaccharide, a carbohydrate, a protein, a dextran, a fat, a liposome, derivatives thereof or combinations thereof.

40. The method of claim 28 wherein said core comprises nanoparticles covered by sheath material.

41. The method of claim 28 wherein a biocompatible iron compound is used in conjunction with the core of at least a biologically acceptable copper compound.

42. The method of claim 41 wherein said biocompatible iron compound is selected from the group consisting of iron hydroxide, iron oxyhydroxide and iron oxide.

43. The method of claim 28 wherein said biologically acceptable insoluble iron compound is selected from the group consisting of iron hydroxide, iron oxyhydroxide and iron.
